(19) United States
(12) Patent Application Publication
(10) Pub. No.: US 2007/0043292 A1
(43) Pub. Date: Feb. 22, 2007

(54) Method for acquiring and evaluating vascular examination data

(75) Inventors: Estelle Camus, Erlangen (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/500,536

(22) Filed: Aug. 8, 2006

(30) Foreign Application Priority Data
Aug. 8, 2005 (DE) 10 2005 037 427.1

(51) Int. Cl. A61B 8/00

(52) U.S. Cl. 600/443

(57) ABSTRACT

The invention relates to a method for acquiring and evaluating vascular examination data, comprising: acquisition of IVUS images of a vessel to be examined using an IVUS catheter; simultaneous acquisition of angiography data of the IVUS catheter having at least one angiography marker; acquisition of OCT images of the same point of the vessel to be examined using an OCT catheter; simultaneous acquisition of angiography data of the OCT catheter having at least one angiography marker; registering the IVUS and OCT images; determination of contours of the structures of the vessel under examination based on the OCT images; arithmetic histological analysis of the co-registered IVUS and OCT images using the information about the contours; and display the results.

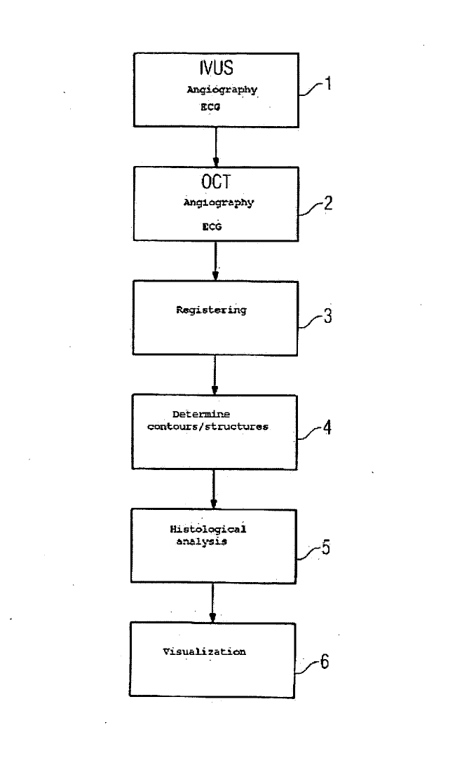

### CROSS REFERENCE TO RELATED APPLICATIONS

This application claims priority of German application No. 10 2005 037 897.8 filed Aug. 10, 2005, which is incorporated by reference herein in its entirety.

### FIELD OF THE INVENTION

The invention relates to a method for acquiring and evaluating vascular examination data.

### BACKGROUND OF THE INVENTION

Methods are already known, with which catheters provided with sensors can be used for vessel imaging. These include ultrasound catheters, for example IVUS (intravascular ultrasound) catheters, which are used to detect atherosclerotic plaque for example. In practice however the automatic evaluation of IVUS images has proven problematic. Such images do indeed show the individual tissue or vessel structures, such as intima, media and plaque layers, but the precise determination and acquisition of contours is difficult and inaccurate, as the structures to be identified are small and the local resolution of the IVUS images is inadequate for this purpose.

In practice therefore contours are input manually to some degree but this is very time-consuming, as the user has to draw in the contours by hand on every image. Also the results depend on the respective user, such that a subsequent quantitative analysis of tissue structures produces different results.

In WO 01/01864 A1 a method is proposed with which IVUS images undergo subsequent processing to determine contours, with image analysis being carried out thereafter. One disadvantage of this is that the local resolution of the IVUS images is limited by the systems used, such that incorrect or inaccurate results are obtained in many instances.

### SUMMARY OF THE INVENTION

The invention therefore has to deal with the problem of creating a method for acquiring and evaluating vascular examination data, which allows a reliable and automatic analysis of image data.

To resolve this problem a method is provided for acquiring and evaluating vascular examination data with the features of the claims.

According to the invention an IVUS catheter is first inserted into a vessel to be examined. At the same time as IVUS images are being acquired, angiography data is also recorded, which shows the IVUS catheter. These x-ray projections can be used later to determine the position of the catheter in the vessel. An OCT catheter is then inserted into the same vessel and moved into the same position the IVUS catheter was in before. The OCT catheter is used to record OCT images and angiography data showing the OCT catheter is also recorded. The IVUS and OCT images are registered; in other words image pairs, which were recorded in the same position in the vessel, are selected from the plurality of recorded images. In the next step contours of the structures of the examined vessel are determined manually and/or automatically using the OCT images. An arithmetic histological analysis of the co-registered IVUS and OCT images is then carried out, based on the information acquired previously about the contours. The images thus analyzed are then displayed.

The invention makes it possible to obtain the data required for an arithmetic histological analysis in a simple manner. As both IVUS images and OCT images are taken into account, it is possible to acquire anatomical structures and any atherosclerotic plaque present with great accuracy and process them automatically. As this information is obtained for every point in the vessel being examined, the diagnostic value of the inventive method is particularly high.

It has proven particularly favorable for an ECG signal to be recorded during acquisition of the IVUS and/or OCT images. The ECG data can be used to acquire or select the IVUS and OCT images recorded in the same cardiac phase. The same applies to the angiography images, to which an ECG signal is also assigned. During the registering of the IVUS and OCT images provided for in step e) images are respectively selected which were recorded during the same cardiac phase.

To simplify implementation of the inventive method and improve its reliability, provision can be made for the position of the catheter(s) to be recorded using a position sensor during acquisition of the IVUS and/or OCT images. Such a position sensor can be integrated in the respective catheter, such that the current position of the OCT or IVUS sensor is acquired automatically. During the examination it is possible to display the current position of the catheter such that both catheters can be moved to precisely the same position one after the other.

In a further embodiment of the method provision can be made for the contours acquired in method step f) to be transferred into the IVUS image(s). The relevant image information is thus taken from the IVUS and OCT images and merged for the subsequent evaluation. In the simplest instance the two images are superimposed. The resulting overall image contains the characteristic features of the IVUS and OCT image.

As an alternative to the position sensor described above, the IVUS catheter and the OCT catheter can each have at least one angiography marker that is visible on an x-ray projection. As the angiography markers are visible on the x-ray projections, they are suitable for determining the position of the catheter.

It is also within the scope of the invention to adjust the image pair comprising the OCT image and the IVUS image manually or semi-automatically or automatically after registering. Parameters such as orientation, enlargement or pixel shift are defined in the context of this adjustment. Manual adjustments are carried out by the user for every image pair. In the case of semi-automatic adjustment the user carries out the adjustment for the first image pair and the parameters used in this process are then also applied to all further image pairs. In the case of automatic adjustment, the contours of rough vessel structures are acquired automatically for all images by segmentation, after which image pairs are processed, for example rotated, elongated and displaced, such that they are superimposed in an optimum manner.

Provision can also be made for the contours of the vessel structures in step f) either to be input manually on the images or to be acquired by means of an automatic image processing method. A combination of both methods is also possible, in that the contours are marked by the user by hand on the first image and then determined automatically using an arithmetic method for all further images.

The inventive method can be further simplified by using a combined IVUS-OCT catheter, such that method steps a) and c) as well as b) and d) can be carried out simultaneously. This can be an integrated catheter, having an IVUS sensor and an OCT sensor, or the combined IVUS-OCT catheter can have a main catheter for separate IVUS and OCT catheters.

### BRIEF DESCRIPTION OF THE DRAWINGS

Further advantages and details of the invention are described below based on an exemplary embodiment with reference to the figure.

The FIGURE shows a flow diagram of the most important steps of the inventive method.

### DETAILED DESCRIPTION OF THE INVENTION

In step 1 the IVUS catheter is inserted into the vessel to be examined. IVUS images and x-ray projections (angiography data) are then recorded at the same time. These two recordings are ECG-triggered, such that both the angiography data and the IVUS images are recorded at the same point of the cardiac cycle. It is ensured with these recordings that the IVUS catheter is clearly visible on the x-ray projections. It is possible to record whole sections of vessels using the pull-back method.

The method can also be configured such that catheters with one or more position sensors are used, such that it is possible to assign the recorded images based on the catheter positions that are recorded at the same time.

After the IVUS catheter has been removed, in step 2 an OCT catheter is inserted into the same vessel and moved into the same position the IVUS catheter was in before. ECG triggering is used in the same manner to record angiography data at the same time as the OCT images. In step 2 it is ensured that the setting of the x-ray unit corresponds to the setting in step 1.

Registering 3 of the IVUS and OCT images then takes place. Registering allows OCT and IVUS images to be selected that show the same layer in the vessel under examination. Data acquired simultaneously by the position sensors is used to this end and compared to assign pairs of images, one OCT image and one IVUS image, to each other. This requires the positions of the two catheters or the position of the respective sensors to be identical or within a predefined tolerance. This tolerance is a function of the characteristics of the OCT and IVUS measuring methods and can be specified by the user.

In one variant of the method the two catheters have one or more angiography markers, which are visible on the x-ray projections. The user then considers the respective position of the IVUS or OCT catheter in relation to the angiography markers on the x-ray projections and can thereby assign the matching pairs of IVUS and OCT images to each other. This method step of assignment and the acquisition of the position of the IVUS and OCT catheters in relation to the angiography markers on the x-ray projections are carried out arithmetically by means of an image processing program with a corresponding algorithm. Using this algorithm allows an OCT image and an IVUS image respectively to be assigned to each other as a pair.

After registering the image pairs are adjusted as required in respect of orientation, enlargement and optionally pixel shift, so that they can subsequently be superimposed. In the simplest instance such adjustment takes place manually with the user adjusting every image pair “by hand”, for example with the aid of a graphic program.

In the case of semi-automatic adjustment the first image pair is adjusted manually with parameters such as orientation, enlargement and pixel shift being defined and said parameters then being used for all further image pairs.

In the case of automatic adjustment the contours of the rough vessel structures, for example the course of the internal wall of the vessel and the lumen, are automatically acquired by segmentation for all images and the image pairs are then processed such that they can be superimposed in an optimum manner. Processing thereby comprises image manipulation such as rotation, elongation and displacement. The parameters for the adjustment, namely orientation, enlargement and pixel shift are either recalculated for each new image pair or defined just once and then used for all the other image pairs.

In step 4 the contours and structures of the vessel under examination are acquired and determined. The OCT images clearly show the contours of the structures, for example the individual vessel layers, such as intima, media or structural changes, such as a lesion, due to the high local resolution of the OCT method. The contours can either be marked by the user manually on every image or they can be acquired automatically using an image processing program and a corresponding algorithm. These methods can also be combined, in that the contours are marked manually by the user on the first image of a series of images and are then acquired automatically using an image processing program on the remaining images, based on the contours input manually on the first image.

Contour determination is followed by a histological analysis 5. Such a method is known as “virtual histology” and is described for example in U.S. Pat. No. 6,200,268 B1. This method can be used for an automatic evaluation of IVUS images, with IVUS images being compared with corresponding values from a database.

The results obtained using the virtual histology method previously carried out are then visualized 6. These results are superimposed with the recorded IVUS images or the recorded OCT images. Superimposition with the OCT images is possible, as these were registered beforehand with the IVUS images. Transparent colors are used so that different data can be shown or hidden in a continuous manner.

Individual method steps of this method can be modified. Blood pressure measurements can be taken instead of the ECG data, with these measurements replacing the ECG data, such that the OCT and IVUS images are triggered by the blood pressure signal.

In addition to the virtual histology analysis of the IVUS images, a similar arithmetic analysis method can be carried out with the OCT data, for example an elastography method.

Instead of using two separate catheters, it is possible to use an integrated IVUS-OCT catheter, such that it is no longer necessary to register the position of the catheter in the vessel and then register separate IVUS and OCT images. It is then only necessary to calibrate the integrated catheter once. In another variant a common main catheter can be used, which contains separate IVUS and OCT catheters. This also makes it simpler to register the position in the vessel under examination and then to adjust the images.

1-8. (canceled)

9. A method for evaluating vascular examination data of a patient, comprising:
acquiring a plurality of IVUS images of a vessel of the patient to be examined using an IVUS catheter;
simultaneously acquiring angiography data of the IVUS catheter having an angiography marker;
acquiring a plurality of OCT images of the vessel using an OCT catheter;
simultaneously acquiring angiography data of the OCT catheter having another angiography marker;
registering a pair of IVUS and OCT images each selected form the IVUS and OCT images which are recorded at an identical position based on the associated angiography data of the catheters;
determining a structure contour of the vessel based on the OCT image;
analyzing the registered IVUS and OCT images based on the contour; and
displaying the analyzing result.

10. The method as claimed in claim 9, wherein the determination of the contour is performed manually or automatically by an image processing method.

11. The method as claimed in claim 9, wherein the analyzing is performed by an arithmetically histological method or an elastrography method.

12. The method as claimed in claim 9, wherein an ECG or blood pressure signal of the patient is recorded during the acquisitions of the IVUS and OCT images.

13. The method as claimed in claim 9, wherein the positions of the IVUS and OCT catheters are recorded by position sensors arranged on the catheters.

14. The method as claimed in claim 9, wherein the contour is transferred into the IVUS images.

15. The method as claimed in claim 9, wherein a parameter of the image pair is adjusted manually after registering.

16. The method as claimed in claim 15, wherein the parameter of the image pair is adjusted semi-automatically by manually adjusting the parameter of a first pair and applying the adjusted parameter to a further image pair after registering.

17. The method as claimed in claim 15, wherein the parameter of the image pair is adjusted automatically by segmentation after registering.

18. The method as claimed in claim 15, wherein the parameter is selected from the group consisting of: orientation, enlargement, and pixel shift.

19. The method as claimed in claim 9, wherein a combined IVUS-OCT catheter is used so that the IVUS and OCT images and the associated angiography data of the catheters are carried out simultaneously and the step of registering is deleted.

20. The method as claimed in claim 19, wherein the combined IVUS-OCT catheter has a common main catheter which comprises separate IVUS and OCT catheters.

21. A medical device for evaluating vascular examination data of a patient, comprising:
an IVUS catheter arranged with a position sensor that records a plurality of IVUS images of a vessel of the patient;
an OCT catheter arranged with another position sensor that records a plurality of OCT images of the vessel containing a contour of the vessel structure;
a computing device that registers a pair of IVUS and OCT images each selected form the IVUS and OCT images which are recorded at an identical position and analyzes the registered images based on the contour; and
a display device that displays a result of the analyzing.
